(19) United States
(12) Patent Application Publication
(10) Pub. No.: US 2003/0060758 A1
(43) Pub. Date: Mar. 27, 2003

(54) Safety syringe for ergonomically coupling and inclinedly retracting used needle device into syringe cylinder

(76) Inventor: Wen-Chin Lu, Taipei (TW)

Correspondence Address: Wen-Chin Lu, P. O. Box 55-846, Taipei, 104, TW

(21) Appl. No.: 09/961,544

(22) Filed: Sep. 21, 2001

(51) Int. Cl. A61M 5/00

(52) U.S. Cl. 604/110; 604/195

(57) ABSTRACT

A safety syringe includes: a plunger slidably held in a syringe cylinder having a flexible coupling rod normally longitudinally formed at a central portion of the plunger, and a needle device detachably secured in a front portion of the syringe cylinder having a tilting hole inclinedly formed in a rear portion of the needle device, whereby upon finishing the injection of the syringe by engaging the flexible coupling rod with the tilting hole to couple the used needle device with the plunger, and upon a rearward retraction of the plunger with the coupled needle device into the syringe cylinder, the flexible coupling rod will automatically resiliently restore to inclinedly bias the coupled needle device within the cylinder to prevent outward protruding of the used needle device for safety protection.

### BACKGROUND OF THE INVENTION

A conventional safety syringe such as a “Simplified Safety Syringe with Retractable Self-biased Needle” of U.S. Pat. No. 5,328,475 may inclinedly retract a used needle into a syringe cylinder by engaging an arrowhead portion 301 of a coupling member 30 formed on a plunger 31 with a biasing socket 27 (27a) recessed in a rear portion of a needle device 2. Whenever coupling the arrowhead portion 301 of the plunger into the biasing socket 27 in the needle device 2, it will require a “great” force to pass the arrowhead portion 301 through a narrow throat area between the port 231 and the conical-shaped socket 27 in order to be inserted into the socket 27, thereby “transferring” such a “great” force when coupling the arrowhead portion 301 with the socket 27 to the patient's body to cause his or her painful feeling.

The present inventor has found the drawbacks of the conventional safety syringe and invented the present safety syringe which can be ergonomically operated.

### SUMMARY OF THE INVENTION

The object of the present invention is to provide a safety syringe including: a plunger slidably held in a syringe cylinder having a flexible coupling rod normally longitudinally formed at a central portion of the plunger, and a needle device detachably secured in a front portion of the syringe cylinder having a tilting hole inclinedly formed in a rear portion of the needle device, whereby upon finishing the injection of the syringe by engaging the flexible coupling rod with the tilting hole to couple the used needle device with the plunger, and upon a rearward retraction of the plunger with the coupled needle device into the syringe cylinder, the flexible coupling rod will automatically resiliently restore to inclinedly bias the coupled needle device within the cylinder to prevent outward protruding of the used needle device for safety protection.

### BRIEF DESCRIPTION OF THE DRAWINGS

FIG. 1 is an illustration showing the plunger means separated from the syringe means secured with the needle device in accordance with the present invention.

FIG. 2 is an illustration when finishing the injection of the present invention.

FIG. 3 is an illustration showing an automatic tilting of a used needle of the present invention.

FIG. 4 shows the ratchet teeth formed on the plunger means of the present invention.

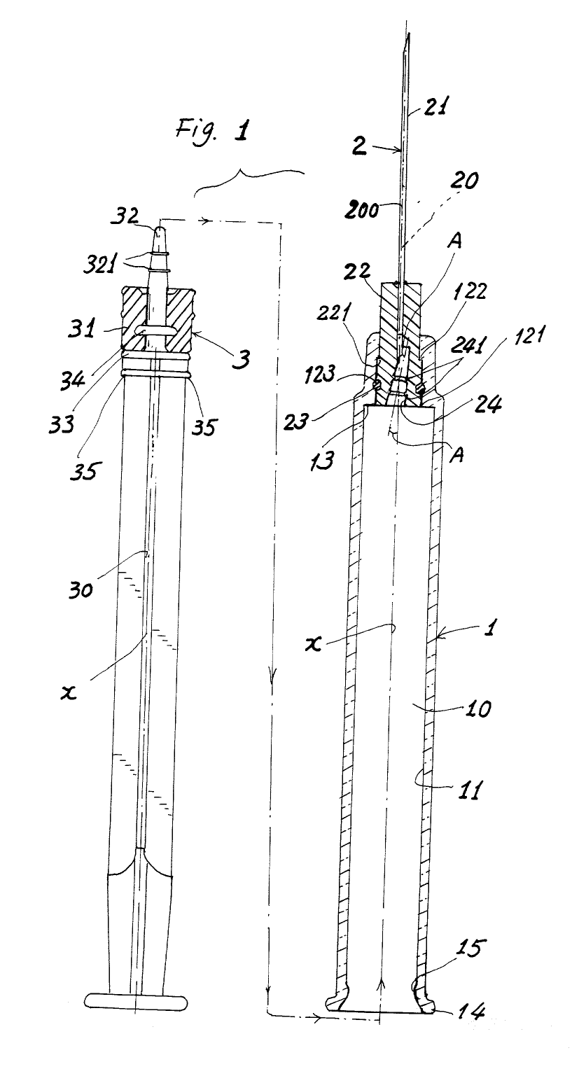

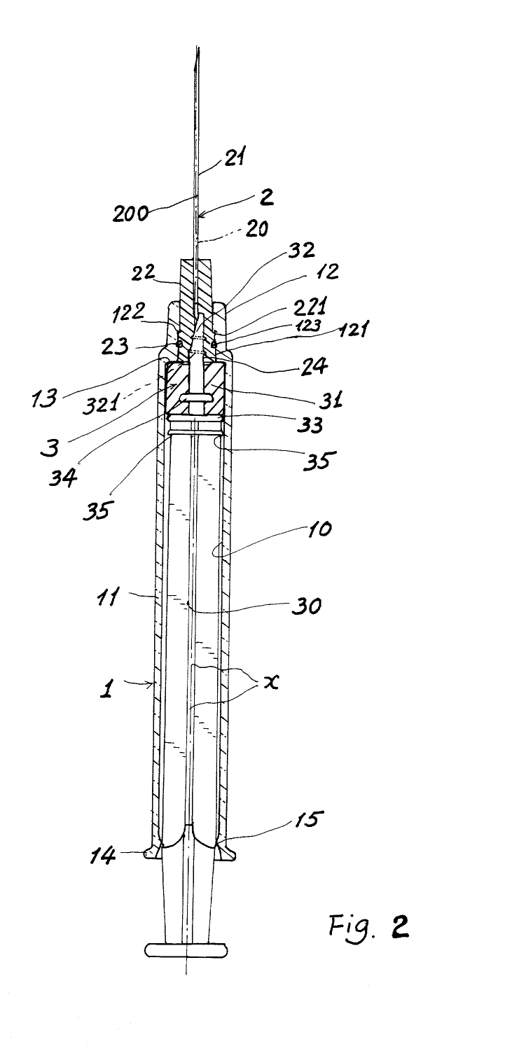

### DETAILED DESCRIPTION

As shown in the drawing figures, the present invention comprises: a syringe means 1, a needle device 2 detachably secured on the syringe means 1, and a plunger means 3 slidably held in the syringe means 1.

The syringe means 1 includes: a syringe cylinder 11 having an interior 10 for filling liquid medicine therein, a sleeve portion 12 formed on a front portion of the cylinder 11, a shoulder portion 13 formed in between the cylinder 11 and the sleeve portion 12, a rear flange (or handle) 14 formed on a rear end portion of the cylinder 11, and an annular extension 15 annularly formed on an inside wall of the cylinder adjacent to the rear flange 14.

The sleeve portion 12 includes: a sleeve hole 121 longitudinally formed through the sleeve portion 12, an annular seat portion 122 annularly formed in a front portion of the sleeve hole 121, and an annular groove 123 annularly recessed in the sleeve hole 121.

The needle device 2 includes: a hollow needle 21 having a needle hole 20 formed through the needle 21, a shank portion 22 secured with the hollow needle 21, a packing ring 23 annularly retained on the shank portion 22 and engaged with the annular groove 123 recessed in the sleeve portion 12, and a tilting hole 24 inclinedly formed in a rear portion of the shank portion 22 having an axis 240 formed at a center of the tilting hole 24 defining an acute angle A between the axis 240 and a longitudinal axis X of the syringe cylinder 11 which is coaxial with the needle axis 200 of the needle 21, with the tilting hole 24 fluidically communicated with the needle hole 20 in the needle 21.

The shank portion 22 is further formed with an annular shoulder portion 221 to be engaged with the annular seat portion 122 formed in the sleeve portion 12 to retard a forward releasing of the shank portion 22 from the sleeve portion 12 when subjected to the liquid pressure during the injection.

The tilting hole 24 includes a plurality of annular engaging portions 241 concentrically formed on (or in) the hole 24 to be engaged with a plurality of rings 321 concentrically formed on a flexible coupling rod 32 of the plunger means 3 which will be further described hereinafter.

The annular engaging portion 241 may be a ring groove annularly recessed in the tilting hole 24, or at least a ratchet tooth annularly formed on an inside wall of the tilting hole 24.

However, the number of the annular engaging portions 241 may be one or two preferably, but not limited in this invention. The tilting hole 24 should have an enough depth in order to snugly engage the elongate coupling rod 32 of the plunger means 3 for a reliable coupling of the needle device 2 with the plunger means 3 after finishing the injection.

The plunger means 3 includes: a plunger 31 secured on a front end portion of a plunger rod 30, a flexible coupling rod 32 made of flexible elastic materials having a proper elasticity and rigidity and protruding forwardly from the plunger 31 to be slightly tapered forwardly and normally restoring forwardly to by coaxially aligned with the longitudinal axis X defined at a longitudinal center of the syringe cylinder 11 when the plunger means 3 is held in said syringe cylinder 11, a circular disk 33 perpendicularly formed on a front portion of the plunger rod 30 for retaining the plunger 31 which is also engaged on an engaging head portion 34 formed on a front portion of the circular disk 33.

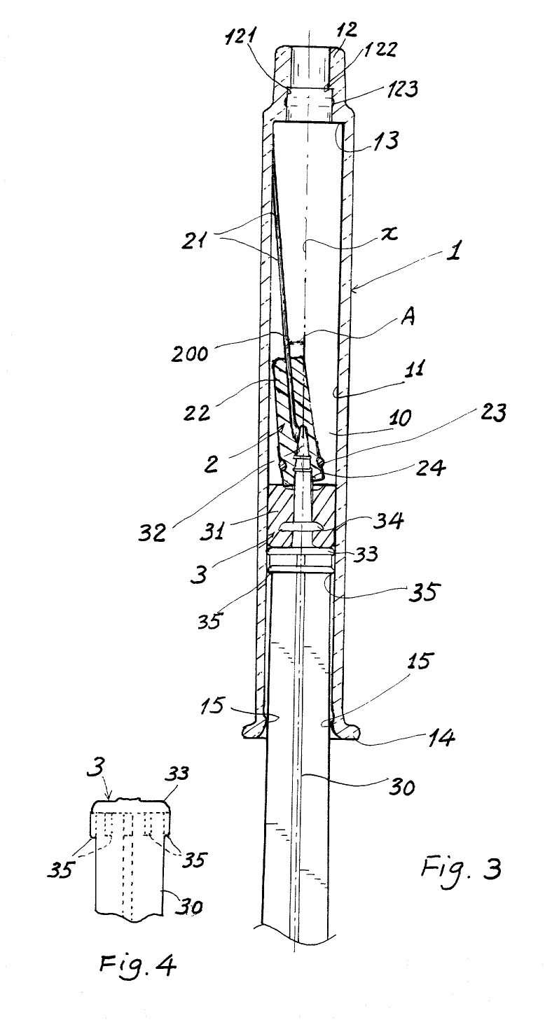

The flexible coupling rod 32 includes a plurality of rings 321 concentrically formed on the rod 32 for snugly engaging the annular engaging portions 241 concentrically formed in the tilting hole 24 when the flexible coupling rod 32 is inserted into the titling hole 24, whereby upon retraction of the plunger 31 as coupled with the needle device 2 into the syringe cylinder 11, the used needle device 2 will be tilted automatically as shown in FIG. 3.

The principles of the present invention are explained and described in detail as follows:

Upon finishing of the injection, the flexible coupling rod 32 is inserted into the tilting hole 24 in the shank portion 22 of the needle device 2 by gradually “bending” the coupling rod 32 into the tilting hole 24 to thereby store the elastic energy of the rod 32.

After retracting the plunger 31 coupled with the needle device 2 into the cylinder 11, the flexible coupling rod 32 will restore by its self resilience or elasticity to be longitudinally aligned with the axis X of the cylinder 11.

However, since the tilting hole 24 of the needle device 2 is snugly engaged with the coupling rod 32 which is now longitudinally restored as shown in FIG. 3, the shank portion 22 and the complete needle device 2 will then be tilted from the axis X for an inclined angle, i.e. the acute angle A.

The used needle 21 will not be further protruded outwardly as retarded against the shoulder portion 13 in the syringe, thereby safely protecting the persons who may contact the syringes.

The present invention is superior to the prior art such as U.S. Pat. No. 5,328,475 with the following advantages:

1. The coupling rod 32 is elongate and slightly tapered to be smoothly snugly engaged (coupled) with the tilting hole 24 in the needle device 2, without causing a great coupling force when coupling the plunger 31 with the needle device 2 and thereby preventing painful feeling of a patient as being injected.

2. After retracting the used needle 21 as coupled with the plunger 31 into the cylinder 11, the used needle will be “precisely” biased inclinedly because a reliable coupling between the coupling rod 32 and the tilting hole 24 having a longer distance and bigger area existing in the frictional engagement (coupling) between the rod 32 and the hole 24.

3. This invention is especially suitable for making small-volume syringe such as 1 cc syringe. The coupling rod 32 can be manufactured more easily since it is not a conventional conical-shaped arrowhead structure which will be made more difficultly when the size of the syringe becomes smaller.

The plunger means 3 (FIGS. 3, 4) further includes: at least a ratchet tooth 35 formed on a rear portion of the circular disk 33 formed on the front portion of the plunger rod 30, whereby upon a rearward pulling of the plunger rod 30, the ratchet tooth 35 will be retarded (or locked) against the annular extension 15 for preventing a rearward releasing of the plunger means 3, once coupled with the used needle device 2, from the cylinder 11.

The ratchet tooth (or teeth) 35 as recessed or formed in a rear portion of the circular disk 33 of the plunger means 3 can be plural ratchet teeth 35 (the number and shapes of teeth being not limited) circumferentially formed in a front portion of the plunger rod 30 as shown in FIG. 3 or may be formed as an annular ratchet tooth 35 annularly formed on a front portion of the plunger rod 30 as shown in FIG. 4.

An arcuate front edge portion may be formed on the circular disk 33 adjacent the front end portion of the plunger rod 30 in order to be smoothly inserted into the interior 10 of the syringe cylinder 11 by passing through the annular extension 15 when assembling the plunger means 3 in the cylinder 11.

The present invention may be modified without departing from the spirit and scope of the present invention.

I claim:

1. A safety syringe comprising:
a syringe means including a syringe cylinder for filling liquid medicine therein and defining a longitudinal axis at a longitudinal center of said cylinder;
a needle device detachably secured in a front portion of said syringe cylinder having a tilting hole inclinedly formed in a rear portion of said needle device; and
a plunger means having a plunger secured on a plunger rod slidably held in said syringe cylinder, having a flexible coupling rod formed on a front portion of said plunger and normally longitudinally aligned with said longitudinal axis of said cylinder, whereby upon finishing an injection of the liquid medicine in said cylinder to engage said flexible coupling rod with said tilting hole to couple said needle device (as used) with said plunger, and upon a retraction of said plunger coupled with said needle device into said cylinder, said flexible coupling rod will automatically resiliently restore to inclinedly bias said needle device within said cylinder to prevent an outward protruding of said needle device.

2. A safety syringe according to claim 1, wherein said syringe means includes: said syringe cylinder having an interior for filling the liquid medicine therein, a sleeve portion formed on a front portion of the cylinder, a shoulder portion formed in between the cylinder and the sleeve portion, a rear flange formed on a rear end portion of the cylinder, and an annular extension annularly formed on an inside wall of the cylinder adjacent to the rear flange.

3. A safety syringe according to claim 1, wherein said needle device includes: a hollow needle having a needle hole formed through the needle, a shank portion secured with the hollow needle, a packing ring annularly retained on the shank portion and engaged with an annular groove recessed in a sleeve portion formed on said cylinder, and a tilting hole inclinedly formed in a rear portion of the shank portion for engaging said flexible coupling rod having an axis formed at a center of the tilting hole and defining an acute angle between the axis and said longitudinal axis of the syringe cylinder which is coaxial with a needle axis of the needle, with the tilting hole fluidically communicated with the needle hole in the needle.

4. A safety syringe according to claim 3, wherein said tilting hole in said shank portion includes a plurality of annular engaging portions concentrically formed on an inside wall of the tilting hole to be engaged with a plurality of rings concentrically formed on said flexible coupling rod of the plunger means.

5. A safety syringe according to claim 4, wherein said annular engaging portion is selected from: a ring groove annularly recessed in the tilting hole, and at least a ratchet tooth annularly formed on an inside wall of the tilting hole.

6. A safety syringe according to claim 1, wherein said plunger means includes: said plunger secured on a front end portion of said plunger rod, said flexible coupling rod made of flexible elastic materials and protruding forwardly from the plunger to be slightly tapered forwardly and normally restoring forwardly to by coaxially aligned with the longitudinal axis defined at the longitudinal center of the syringe cylinder when the plunger means is held in said syringe cylinder, a circular disk perpendicularly formed on a front portion of the plunger rod for retaining the plunger which is engaged on an engaging head portion formed on a front portion of the circular disk.

7. A safety syringe according to claim 6, wherein said plunger means further includes: at least a ratchet tooth formed on a rear portion of the circular disk formed on the front portion of the plunger rod, whereby upon a rearward pulling of the plunger rod, the ratchet tooth will be retarded against an annular extension formed in the cylinder for preventing a rearward releasing of the plunger means from the cylinder when the plunger means is coupled with the needle device as used.

8. A safety syringe according to claim 7, wherein said ratchet tooth is an annular ratchet tooth annularly formed on a front portion of the plunger rod.
